(19) United States
(12) Patent Application Publication
(10) Pub. No.: US 2019/0083749 A1
(43) Pub. Date: Mar. 21, 2019

(54) SURGICAL DEVICE TIP WITH ARC LENGTH VARYING CURVATURE

(71) Applicant: Vanderbilt University, Nashville, TN (US)

(72) Inventors: Philip J. Swaney, Nashville, TN (US); Peter York, Somerville, MA (US); Hunter B. Gilbert, Nashville, TN (US); Robert J. Webster, III, Nashville, TN (US); Arthur W. Mahoney, Nashville, TN (US); Patrick Wellborn, Nashville, TN (US)

(21) Appl. No.: 16/176,068

(22) Filed: Oct. 31, 2018

Related U.S. Application Data
(62) Division of application No. 15/164,257, filed on May 25, 2016.
(60) Provisional application No. 62/166,310, filed on May 26, 2015.
(60) Provisional application No. 62/296,620, filed on Feb. 18, 2016.

(51) Int. Cl. A61M 25/01; A61B 17/34; A61B 17/00; A61B 34/00; A61B 34/30; A61B 34/35

(52) U.S. Cl. CPC A61M 25/0138; A61B 17/3417; A61B 17/00234; A61B 2017/00309; A61B 2017/00323; A61B 17/3478; A61B 2017/00991; A61B 34/71; A61B 2034/301; A61B 34/35; A61M 25/0147; A61M 25/0113

(57) ABSTRACT

The needle-sized surgical tools used in arthroscopy, otolaryngology, and other surgical fields could become even more valuable to surgeons if endowed with the ability to navigate around sharp corners to manipulate or visualize tissue. A needle-sized bendable joint design that grants this ability. It can be easily interfaced with manual tools or concentric tube robots and is straightforward and inexpensive to manufacture. The bendable joint includes of a nitinol tube with several asymmetric cutouts, actuated by a tendon.

### RELATED APPLICATIONS

This application is a divisional of U.S. patent application Ser. No. 15/164,257, filed May 25, 2016, which claims the benefit of U.S. Provisional Application Ser. No. 62/166,310, filed May 26, 2015, and U.S. Provisional Application Ser. No. 62/296,620, filed Feb. 18, 2016. The subject matter of these applications is hereby incorporated by reference in its entirety.

### GOVERNMENT RIGHTS

This work was funded in part by the National Science Foundation (NSF) under CAREER award U.S. Pat. No. 1,054,331 and three Graduate Research Fellowships. It was also funded in part by the National Institutes of Health (NIH) under award numbers R01 EB017467 and R21 EB017952. The U.S. Government may have certain rights to the invention.

### FIELD OF THE INVENTION

The present invention relates to surgical tools for performing surgical operations. More specifically, the present invention relates to small diameter surgical tools for navigating the patient's anatomy in order to deliver therapy to a target location in the patient's body. In particular, the present invention relates to a surgical device with a bendable joint, such as a bendable tip that has an arc length varying curvature for implementation in small-diameter microsurgical tool devices, such as hand-operated catheter-like manipulators or concentric tube robots.

### BACKGROUND

There is a pressing need in robotic or remotely controlled hand-operated surgery for small-diameter surgical tools with bendable tips, which are sometimes referred to as bendable tip 12 joints or bendable tips 12 because they act as the bending joint proximate to the tool at the distal end or tip much in the manner that the human bendable tip 12 serves in relation to the hand. Most existing small-diameter surgical tools devices do not include bendable tips 12 and thus cannot navigate the sharp corners encountered in surgery, such as those at the skull base, in the middle ear, and in the ankle. Moreover, dexterity driven tasks, such as tissue resection and suturing, can be difficult to perform without a bendable tip 12, especially through the small openings characteristic of natural orifice or percutaneous procedures.

In one particular surgical field, small-diameter bendable tips 12 are needed to augment the capabilities of microsurgical devices, such as small-diameter catheter-like or concentric tube surgical robots, which can have diameters on the needle-sized order (e.g., having diameters as small as 1.0 mm or less). The performance of these small-diameter robotic systems, which are devised for delicate and intricate surgical procedures such as pituitary tumor resection, neurosurgery, and intracardiac surgery, among others, can be significantly enhanced with the addition of small bendable tips 12 for aiding in manipulating their end-effectors.

Many small bendable tips 12 based on traditional mechanical linkages have been devised in the past. For example, previous small bendable tips 12 have been designed to incorporate the use of ball joints, universal joints, cable/pulley mechanisms, lead screws, parallel serial chains, and flexures. These designs range from 2.4 to 15.0 mm. Although it could be possible to downscale each of these designs to some degree, designs with a continuum structure, i.e., those in which are machined or otherwise engineered directly into the shaft structure of the catheter, needle, concentric tube, etc. would be easier to miniaturize than those containing multiple components. Among these continuum structures, those with the fewest components are most desirable for downscalability, making designs that involve machining the shaft of the device itself particularly appealing. Examples of continuum structures involve cutting nitinol tubes to create rectangular or triangular cutouts that form a compliant region for bending. In these instances, however, the diameters remain comparatively large, e.g., on the order of 6-10 mm.

Additionally, known bendable structures suffer from limitations that relate to the manner in which their bending takes place. An example of this is shown in FIG. 13, which illustrates schematically a commercially available bendable tip catheter 300. The catheter 300 is illustrated in various stages of actuation, beginning at a zero actuated condition 302, fully actuated condition 308 and intermediate actuated conditions 304 and 306. As shown in FIG. 13, as the actuation proceeds, the most proximal sections of the catheter 300, indicated generally at 310, bend first, followed by intermediate sections 312 and distal sections 314. As a result, in the fully actuated condition 308, the proximal sections 310 achieve full bend, and the degree of bending is reduced through the intermediate sections 312 and distal sections 314. This behavior can be highly unfavorable in small spaces where these devices can be operated, because a high degree of bending at the distal-most portions is not only desired, but can be critical to performing a successful procedure.

### SUMMARY

A small-diameter bendable tip allows a user to design a tip with arc length varying curvature. The user can select properties, such as which portion of the tip bends first, in which order subsequent portions bend, how far each section is able to bend, and the general motion of the bending). This design improves the performance of commercial bendable catheter tips and microsurgical devices by enabling the user to specify the motion of the bendable tip and enhancing the dexterity of the tip in small spaces during surgery, an important characteristic of small steerable surgical devices.

According to one aspect, the invention relates to a bendable joint including a tubular structure including a tubular side wall that extends along an axis and defines an inner lumen. At least one cutout is positioned along the length of the sidewall. Each cutout includes an axial portion of the sidewall that is removed and provides communication with the inner lumen. Each cutout helps to define a bend joint and at least one bend section. The bend joint includes the remaining portion of the side wall left along the length of the cutout. The at least one bend section includes complete tubular portions of the sidewall on opposite sides of the cutout. Each bend joint can deflect so that adjacent bend sections move relative to each other and assume a curved configuration.

According to another aspect, the bend sections associated with each bend joint an move toward each other in response to deflection of the bend joint.

According to another aspect, the bendable joint can include a tendon cable that extends within the inner lumen and has a connection with a distal one of the bend sections, Tension on the tendon cable can be applied to the distal one of the bend sections, which causes the bend joints proximal of the connection to deflect and causes the associated bend sections to move towards each other and assume a curved configuration.

According to another aspect, the cutouts can have geometries selected such that the physical properties of the bend joints differ from each other, which causes the curvature of the bend joint to vary along its length. The cutouts can have geometries selected such that the physical properties of the bend joints differ from each other, which causes the bend joints to deflect in a predetermined order in response to tension applied to the tendon cable.

According to another aspect, the tubular structure can be an inner tube of a concentric tube robot.

According to another aspect, the cutouts can have rectangular geometries. The cutouts can be aligned with each other along the axis of the tubular structure. The cutouts can be rotated relative to each other along the axis of the tubular structure.

According to another aspect, the geometries of the bend sections defined by the cutouts can be configured to define the amount of deflection that each bend joint can undergo. The geometries of the bend sections defined by the cutouts can be configured to collectively define the range of bending motion that can be achieved by the bendable joint.

According to another aspect, the cutouts can define the joint along a tip portion of the tubular structure.

According to another aspect, the tubular structure can be a nitinol tube.

According to another aspect, the tubular structure can include a needle structure. The terminal end portion of the tubular structure can include a needle tip comprising a sharpened point. A cutout can be positioned adjacent to the needle tip. The bend joint defined by the cutout can allow the tip to deflect relative to the remainder of the tubular structure. The needle tip can include a beveled lead surface that is angled relative to a longitudinal axis of the tubular structure. The lead surface can be configured such that when the needle tip is advanced longitudinally through tissue, the tissue acting on the lead surface urges the needle tip to deflect relative to the remainder of the tubular structure through bending of the bend joint.

According to another aspect, the bendable joint can include a tendon cable that extends within the inner lumen and has a connection with the needle tip. Tension on the tendon cable can be applied to the needle tip, which causes the bend joint adjacent the needle tip to deflect. Deflection of the needle tip relative to the remainder of the tubular structure can cause the tubular structure to follow a curved path when advanced through tissue.

According to another aspect, the bendable joint can include an end effector for performing a surgical function positioned at the distal end of the tubular structure distal of the bend joint. A tendon cable can extends through the tubular structure and be connected to the end effector. The tendon cable can be actuatable to cause actuation of the end effector.

According to another aspect, the cutouts can have non-rectangular geometries. The cutouts can have geometries that are generally key-shaped when viewed in profile. The key-shaped cutout geometries result in the bend sections having a generally tapered configuration, and the bend joints having semicircular edge portions. Adjusting the geometry of a circular portion of the key-shaped cutouts can affect the force required to deflect the bend joints. Adjusting the spacing and angle of tapered edges of the cutouts can affect the range of motion permitted between adjacent bend sections.

### DRAWINGS

FIGS. 1A and 1B illustrate an apparatus including a small diameter bendable tip, according to an example embodiment.

FIGS. 2A-2D illustrate the bending of the small diameter bendable tip portion of the apparatus of FIG. 1.

FIGS. 3A-D illustrates example configurations of the small diameter bendable tip.

FIGS. 4A and 4B are schematic diagrams illustrating certain forces and moments acting on an example configuration of the small diameter bendable tip.

FIG. 5 illustrates certain parameters and relevant kinematic values for a portion of the small diameter bendable tip.

FIGS. 6A-6C illustrate geometric layout and parameters for portions of the small diameter bendable tip.

FIG. 7 is a schematic illustration of a portion of the small diameter bendable tip depicting certain kinematic properties.

FIG. 8 illustrates one example configuration in which an example small diameter bendable tip with a distally mounted surgical instrument.

FIG. 9 illustrates the example configuration of FIG. 8 in different operational positions.

FIG. 10 is a magnified view of a portion of the bendable tip 12 apparatus illustrated in FIGS. 8 and 9.

FIG. 11 is a graph that illustrates a modeled versus experimental spatial trajectories of the example bendable tip 12 apparatus configuration of FIGS. 8-10.

FIG. 12 is a graph that illustrates tendon force versus bendable tip 12 rotation for the example bendable tip 12 apparatus configuration of FIGS. 8-10.

FIG. 13 illustrates a known bendable tube device.

FIGS. 14A and 14B illustrate alternative configurations of a small diameter bendable tip apparatus.

FIGS. 15A and 15B illustrate the operation of an alternative configuration of a small diameter bendable tip apparatus.

FIGS. 16A and 16B illustrate an alternative configuration of a small diameter bendable tip apparatus.

FIGS. 17A-17D illustrate the operation of the small diameter bendable tip apparatus of FIGS. 16A and 16B.

FIGS. 18A and 18B illustrate the operation of the small diameter bendable tip apparatus of FIGS. 16A and 16B featuring an alternative actuator.

### DESCRIPTION

### Device Design

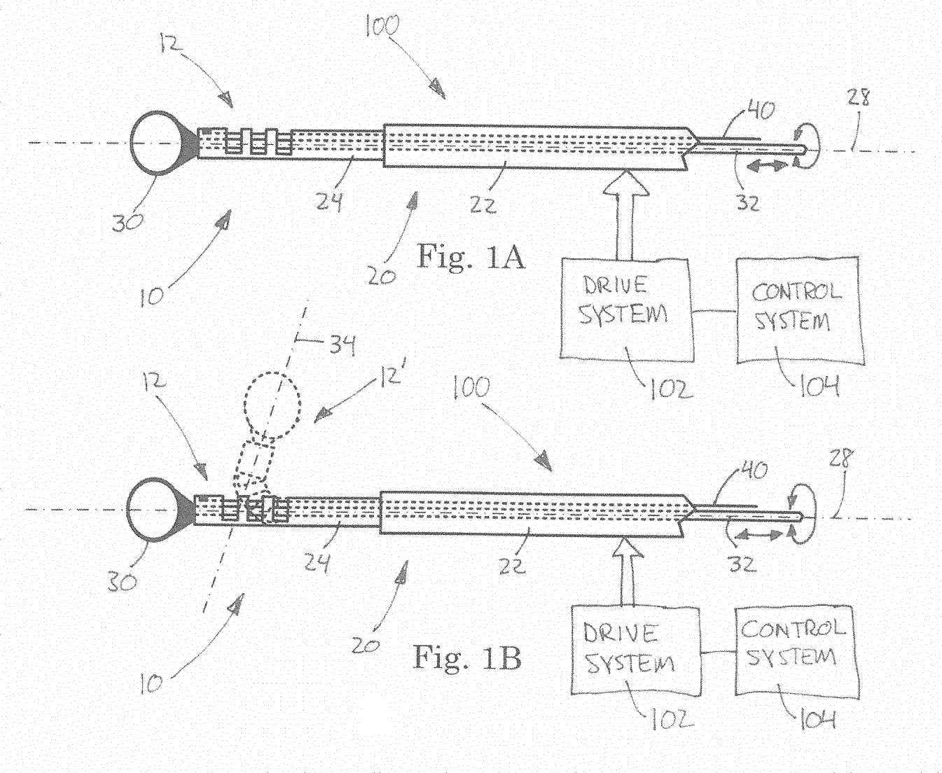

The present invention relates to a surgical device with a bendable tip that has an arc length varying curvature for implementation in small-diameter surgical tool devices. Referring to FIGS. 1A and 1B, according to one example embodiment, a surgical system 100 includes an apparatus 10 in the form of a small-diameter surgical tool device includes a small-diameter bendable joint 12. In the example embodiment of FIGS. 1A and 1B, the bendable joint 12 forms a bendable tip of the apparatus 10 and is therefore referred to herein as a bendable tip 12. The position of the bendable joint, however, is not limited and could be located at any location along the length of the apparatus 10. Additionally, in the example embodiment of FIGS. 1A and 1B, the small-diameter surgical tool 10 comprises a concentric tube robot 20 comprising at least two concentric tubes. The surgical tool 10 could have other configurations, such as a tubular catheter configuration.

As shown in FIGS. 1A and 1B, the surgical system 100 can also include a drive system 102 for actuating the various components of the apparatus 10, and a control system 104 for controlling the actuation system. The drive system 102 includes various actuation components, such as motors, solenoids, actuators, linkages, drive mechanisms, transmissions, etc. that supply the motive forces for operating the apparatus 10. The control system 104 includes the input, processing, and signal generating components that generate the drive signals for controlling operation of the actuation components of the drive system 102.

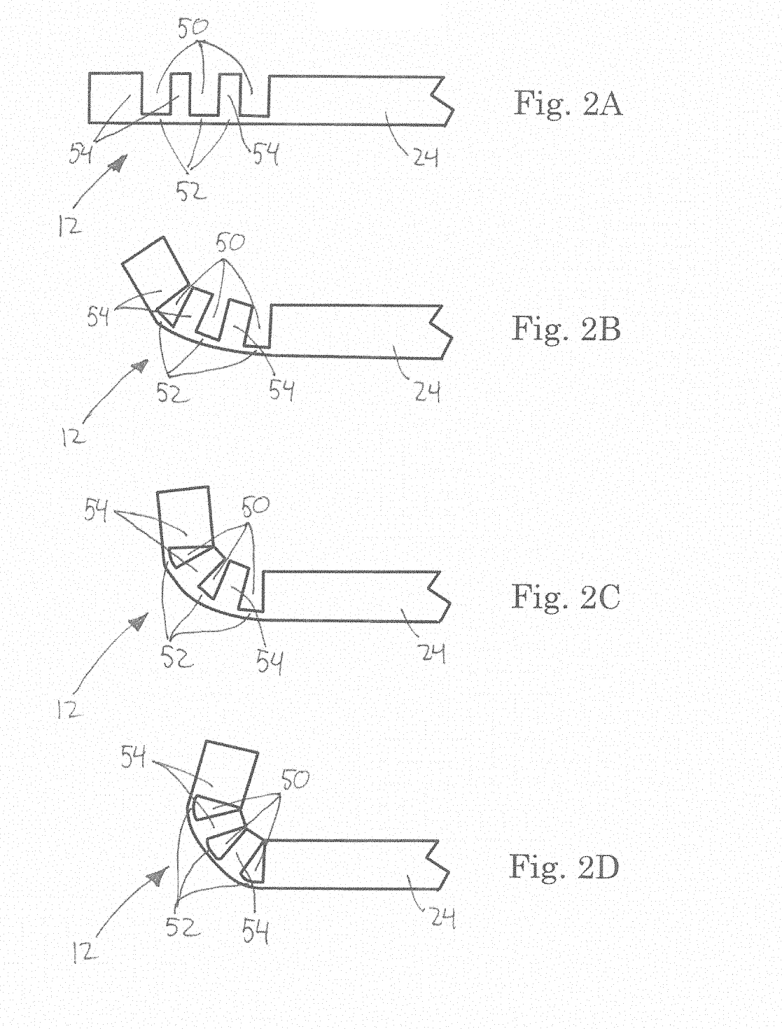

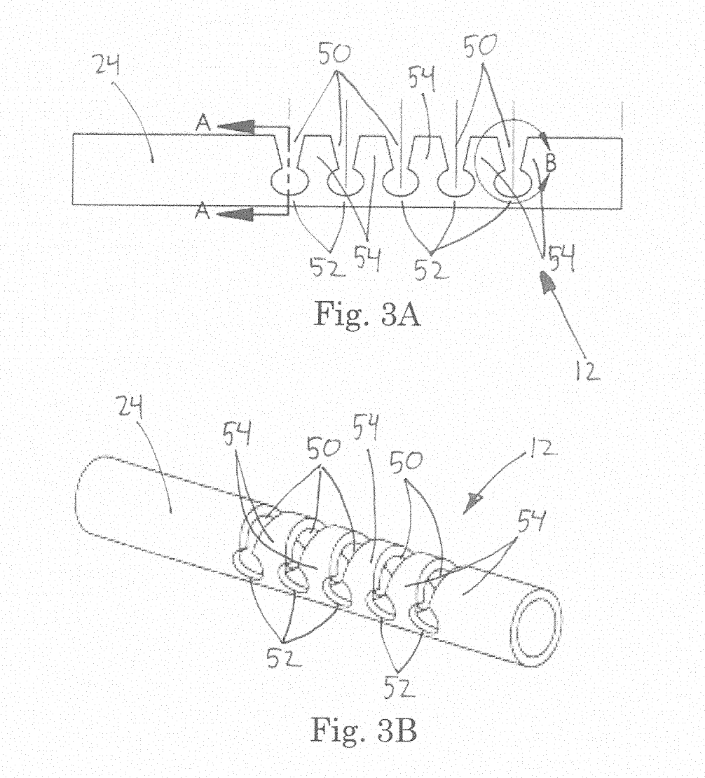

In the example embodiment, there are two concentric tubes: a straight, typically stainless steel, outer tube 22 and a curved, typically nitinol, inner tube 24. The outer tube 22 and inner tube 24 are individually and independently movable both axially along and rotationally about a longitudinal axis 28. In the retracted position illustrated in FIGS. 1 and 2, the inner tube 24 is retracted within the outer tube 22 such that the portion protruding from the outer tube is substantially straight. As known in the art, the inner tube 24 assumes its curved configuration as it is extended or telescoped out of the outer tube 22. This, in combination with the axial and rotational manipulation of the outer and inner tubes 22, 24, defines a work space or volume within which the concentric tube robot 20 can deliver its tip to any location.

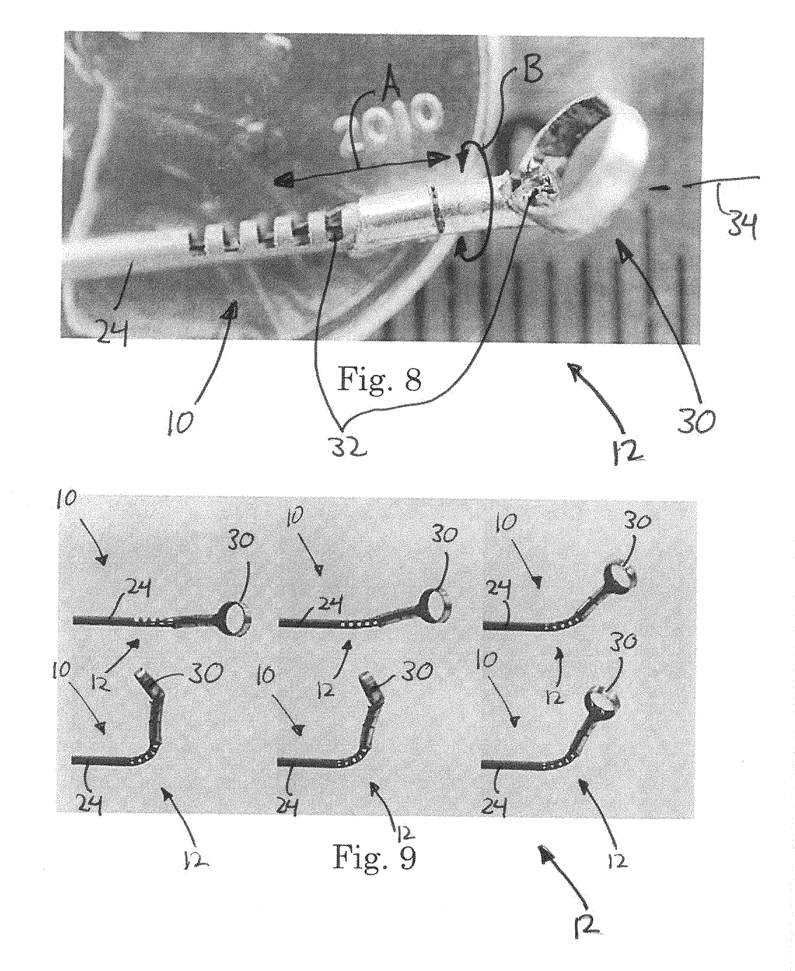

Referring to FIGS. 8 and 9, the surgical tool 10 also includes an end effector or instrument 30 that is located at the distal end of the tool. In the example embodiment, the instrument 30 is a surgical instrument in the form of a curette that is typically used to cut, scrape or otherwise remove tissue. The surgical instrument 30 could, however, be any surgical tool for which delivery via a small-diameter surgical tool 10. For example, the surgical instrument could comprise grippers, surgical lasers, graspers, retractors, scissors, imaging tips, cauterizing tips, ablation tips, morcelators, knives/scalpels, cameras, irrigation ports, suction ports, needles, or any other suitable surgical instrument.

The surgical tool 10 can deliver the surgical instrument 30 to any location within the work space of the concentric tube robot 20. The surgical instrument 30 itself can be further manipulated, for example, via a flexible rod or cable 32 that extends through inner lumens of the concentric tubes 22, 24. The cable 32 can be manipulable, for example, to cause rotation (arrow B in FIG. 8) or linear translation (arrow A in FIG. 8) of the surgical instrument 30 about an instrument axis 34, as indicated generally by arrows in FIGS. 1A and 1B. In FIG. 1A, the instrument axis 34 is coaxial with the tool axis 28. In FIG. 1B, the instrument axis 34 is transverse to the tool axis 28. For the alternative surgical instruments 30, the cable 32 may serve other purposes, such as actuating a mechanical linkage of the instrument or delivering energy to the instrument.

The small-diameter bendable tip 12 is positioned at the distal end of the inner tube 24 just proximal of the instrument 30 and thereby connects the surgical instrument 30 to the concentric tube robot 20. Conveniently, in the example embodiment of FIGS. 1A and 1B, the bendable tip 12 is formed integrally as the distal end portion of the nitinol inner tube 24. The bendable tip 12 has a non-actuated condition, shown in FIG. 1A, in which the bendable tip is not bent and extends essentially or substantially along the tool axis 28. The bendable tip 12 has an actuated condition, shown in dashed lines at 12′ in FIG. 1B, in which the tip is bent along an arc, thus positioning the instrument axis 34 transverse to the tool axis 28. The bendable tip 12 is selectively actuatable to place the tip at any intermediate position along the arc of travel between the actuated and non-actuated positions.

Actuation of the bendable tip 12 is effectuated through the actuation of a tendon cable 40 which extends through the inner lumen of the concentric tubes 22, 24 and is connected to the bendable tip. A motor or other suitable drive mechanism (not shown) applies and varies the tension on the tendon cable 40 in order to effectuate the desired degree of bend in the tip. The drive mechanism for the tendon cable 40 can be integrated into the drive unit that operates the concentric tube robot 20 and the surgical instrument 30. Actuation of the concentric tube robot 20, surgical instrument 30, and bendable tip 12 can thus be controlled via a single controller or control system that integrates and coordinates the control of all of these devices.

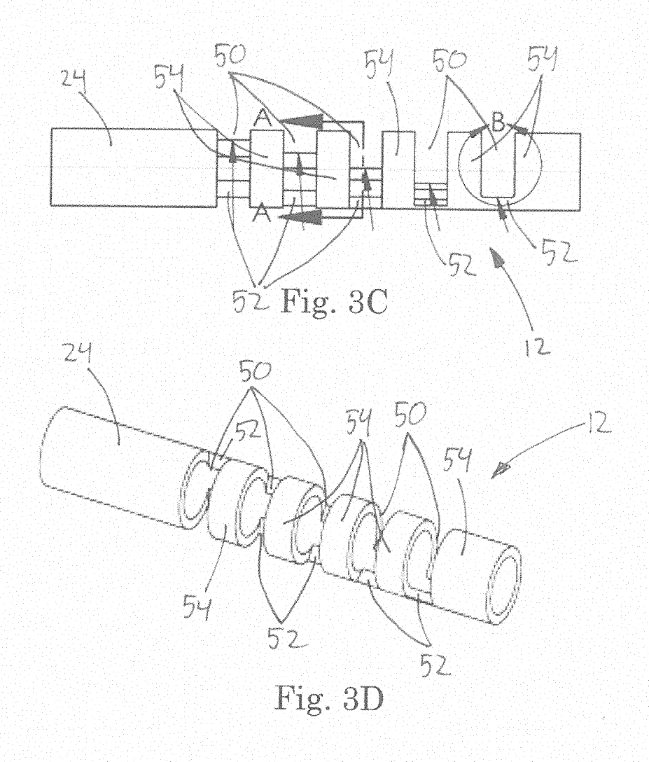

FIGS. 2A-2D illustrate the distal end of the inner tube 24, including the bendable tip 12, in greater detail. In FIGS. 2A-3D, the illustrated bending of the tip 12 is effectuated through the application of tension to the tendon cable, which is omitted from these figures in order to illustrated the structure of the tip in greater detail.

Referring to FIGS. 2A-2D, the bendable tip 12 is formed by a series of cutouts 50 in which tube material (e.g., nitinol) is removed from the inner tube 24. The cutouts 50 define bend joints 52, which are the portions of the inner tube 24 that remain after the removal of the cutout material. The cutouts 50 also define tubular bend sections 54 that extend between the bend joints 52. In the example embodiment illustrated in FIGS. 2A-2D, the bendable tip 12 includes three cutouts 50 that define three bend joints 52 and three bend sections 54. The bendable tip 12 could include a greater number of cutouts 50 or fewer cutouts, depending on factors, such as the desired performance characteristics of the tip and the particular application in which the tip is to be implemented.

As shown in FIGS. 2A-2D, the bendable tip 12 proceeds from a non-actuated or zero bend/deflection condition (FIG. 2A) to a fully actuated, full bend/deflection condition (FIG. 2D). Between these extremes, the bendable tip 12 proceeds through intermediate degrees of bending (FIGS. 2B and 2C). As the tip 12 bends, the bend joints 52 deflect and the bend sections 54 move and rotate/pivot. This movement is blocked when adjacent bend sections 54 engage each other or, in the case of the most proximally located bend section (the rightmost in FIGS. 2A-2D), engage the remainder of the inner tube 24. The degree of movement that each bend section 54 is permitted to undergo is thus defined and limited by the geometry of the cutout(s) 50 that define it.

In the example of FIGS. 2A-2D, the bendable tip 12 includes cutouts 50 that are generally rectangular when viewed in profile, resulting in generally rectangular bend sections 54 and bend joints 52 with flat edge portions. Adjusting the geometry of the cutouts 50 can affect the bending action of the bend joints 52 and bend sections 54. For example, adjusting the depth of the cutouts 50 can alter the cross-section of the bend joints 52 and can thereby affect the force required to deflect the bend joints. Adjusting the width of the cutouts 50 affects the spacing of the bend sections 54, which can affect the range of motion permitted between adjacent bend sections.

Examples of different geometries for the cutouts 50 are illustrated in FIGS. 3A-3D. Note that in FIGS. 3A-3D, the illustrated bend joint 12 includes five cutouts 50, as compared to the three cutouts in the embodiment of FIGS. 2A-2D. Referring to FIGS. 3A and 3B, the bendable tip 12 includes cutouts 50 that are generally key-shaped when viewed in profile, resulting in generally tapered or spade shaped bend sections 54 and bend joints 52 with semicircular edge portions. Adjusting the geometry of the circular portion of the cutouts 50 can affect the force required to deflect the bend joints 52. Adjusting the spacing and angle of the tapered edges of the cutouts 50 can affect the range of motion permitted between adjacent bend sections.

The pattern of the cutouts 50 could be arranged in patterns that differ from the straight line pattern in the example embodiment of FIGS. 1-2. For example, referring to FIGS. 3C and 3D, the bendable tip 12 includes cutouts 50 that are generally rectangular in shape when viewed in profile, and therefore the bend joints 52 and bend sections 54 act in a similar or identical manner to those illustrated and described with respect to the bendable tip of FIGS. 2A-2D. In FIGS. 3C and 3D, however, the cutouts 50 are progressively rotated relative to each other so that the bend joints 52 are arranged in a helical pattern along the length of the bend joint 12 and the bend sections are rotated with respect to each other. This configuration causes the tip 12 to assume a helical shape when deflected, which can provide the tip with an added degree of dexterity. From this, it can be seen that, according to the invention, the pattern and spacing of the cutouts 50 and, thus, the bend joints 52 and bend sections 54 can be selected so that the tip 12 assumes a desired bent configuration when actuated. These patterns are not limited to the linear or helical patterns illustrated in the figures, as those figures are illustrative of possible configurations and are not meant to limit or otherwise restrict other possible configurations. The configuration of the bendable tip 12 can be selected to any desired configuration through the selection of appropriately configured and spaced cutouts 50.

According to the invention, the construction of the bendable tip 12 allows the tip to be designed with an arc length varying curvature that is customizable to meet the demands of the user. By “arc length varying curvature,” it is meant that the properties or characteristics of under which each bend joint 52 and bend section 54 act during bending of the tip 12 can be configured individually. For each bend joint 52 and bend section 54, the geometry of the cutout 50 can be configured to allow a user to select bend characteristics, such as the amount of force required to deflect each bend joint 52, the order in which each bend joint/section of the tip bends, the range of deflection for each bend joint/section, and the general motion, i.e., straight vs. curved/helical, of the bending. The design of the bendable tip 12 offers improved performance by enabling the user to specify the motion of the bendable tip and enhancing the dexterity of the tip in small spaces during surgery.

FIGS. 2A-2D illustrate this construction. For convenience in describing the construction and operation of the bend joint 12, the bend joints 52 and bend sections 54 in FIGS. 2A-2D are referred to as first, second and third as viewed from distal to proximal, i.e., left to right in the figures.

Viewing FIGS. 2A-2D, the bending motion of the of the bendable tip 12 is configured so that the first bend joint 52 deflects first and the first bend section 54 moves/pivots/rotates first. This is shown in FIG. 2B, where the first bend section 54 moves/pivots/rotates into engagement with the second bend section before the second and third bend sections undergo significant movement. This is not to say that there is not some deflection/movement in the second or third bend joints 52 and bend sections 54. Indeed, some deflection or movement in these joints and sections can be expected and is therefore illustrated in FIG. 2B. This deflection, however, is minimal compared to the full deflection of the first bend joint 52.

Referring to FIG. 2C, after the first bend joint 52 undergoes full deflection such that the first bend section 54 engages the second bend section, the second bend joint 52 deflects and the second bend section 54 moves/pivots/rotates into engagement with the third bend section. This occurs while there is minimal deflection in the third bend joint 52. Again, this is not to say that there is not some deflection/movement in the third bend joint 52 and bend section 54. Indeed, some deflection or movement in these joints and sections can be expected and is therefore illustrated in FIG. 2B. This deflection is minimal compared to the full deflection of the second bend joint 52.

Referring to FIG. 2D, after the second bend joint 52 undergoes full deflection such that the second bend section 54 engages the third bend section, the third bend joint 52 deflects and the third bend section 54 moves/pivots/rotates into engagement with the inner tube 24. From this, it can be seen that the bendable tip 12 is configured to function so that the bend sections 54 move in succession, from tip to base, i.e., from distal to proximal. This particular motion can be advantageous, for example, in permitting the apparatus 10 to navigate sharp turns within the patient's anatomy. For instance, in FIGS. 2A-2D, it can be seen that the overall length of the apparatus 10 is shortened as the tip 12 undergoes bending. If, however, the inner tube 24 is controlled to advance axially at the same rate that the length is shortened due to the bending, the net result is that the tip, i.e., the surgical instrument, will navigate a sharp turn that otherwise could not be navigated if all of the bend joints 52 deflect at the same time.

The arc length varying curvature of the bendable tip 12 is customizable through selection of the geometry of the cutouts 50, which define the geometries of the bend joints 52 and bend sections 54. Each cutout 50 can have a uniquely configured geometry that defines the amount of force required to deflect the bend joint 52, the direction in which the joint deflects, and the geometry of the bend sections 54, which define the limit of angular deflection. In this manner, the behavior of each segment of the tip 12, i.e., the bend joint 52 and adjacent bend sections 54 defined by a cutout 50, can be tailored so that the motion profile of the tip, and the attached surgical instrument 30, is suited to perform the desired tasks. The tip 12, so designed, can access the target anatomical structures while avoiding others.

### Alternative Configurations

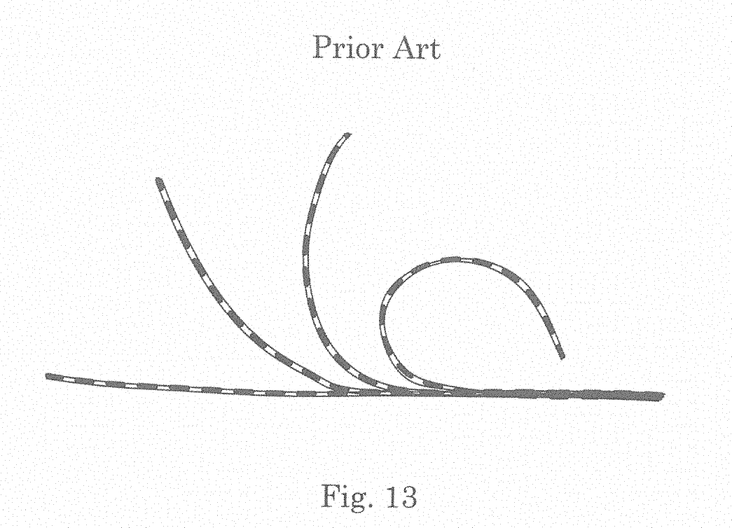

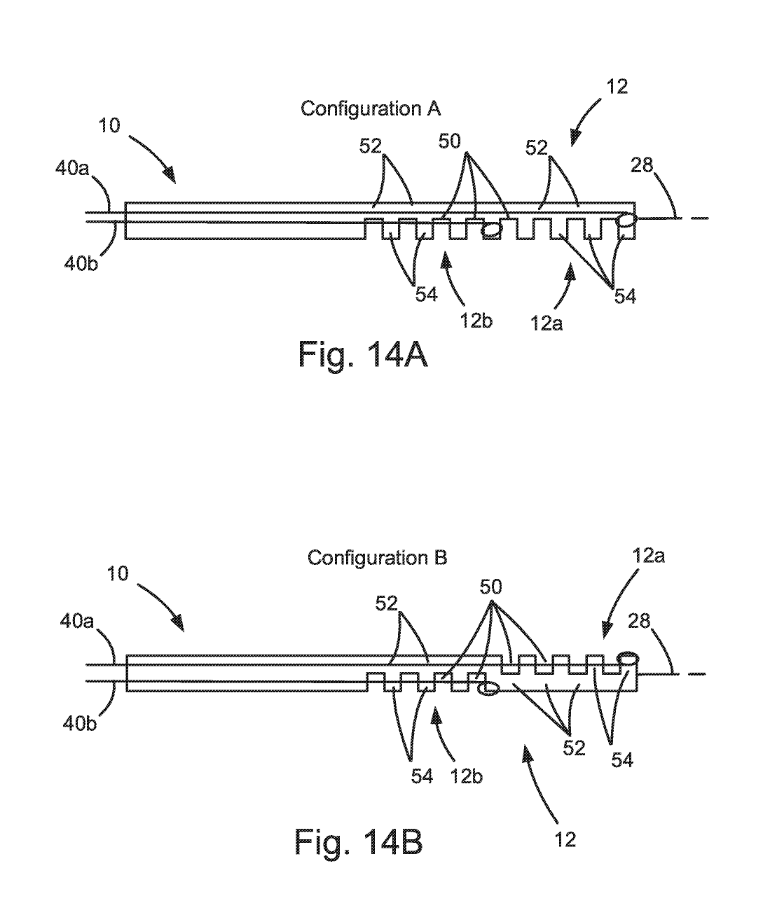

The bendable tip could have additional configurations that lend to its ability to provide a desired degree of reach and dexterity. For example, referring to FIG. 14, the apparatus 10 could include multiple tendon cables 40 that are actuatable independently to effectuate bending of the tip. In the examples of FIG. 14, the apparatus 10 includes two tendon cables 40a, 40b. The apparatus 10 could, however, include a greater number of tendon cables 40.

In Configuration A in FIG. 14, tendon cable 40a is connected to the bend section 54 at the terminal end of the bendable tip 12. The tendon cable 40b is connected to a bend section 54 at about the midpoint of the bendable tip 12. The tendon cable 40b can be actuated to bend a proximal section 12b of the bendable tip 12. The tendon cable 40a can be actuated to bend a distal section 12a of the bendable tip 12. In operation, the tendon cable 40b can be manipulated to bend the proximal section 12b in order to adjust the position and attitude of the distal section 12a, which can then be actuated to complete the task.

Configuration B in FIG. 14 is similar to Configuration A, except that the radial positions of the bend sections 12a and 12b are rotated 180 degrees from each other about the axis 28. In this example configuration, the bend sections 12a and 12b bend in opposite directions, and the tendon cables 40a and 40b are configured to effectuate this bending. Of course, the apparatus 10 could be configured to include multiple bend sections arranged in varying radial positions with corresponding tendon cables providing actuating capabilities for those sections individually.

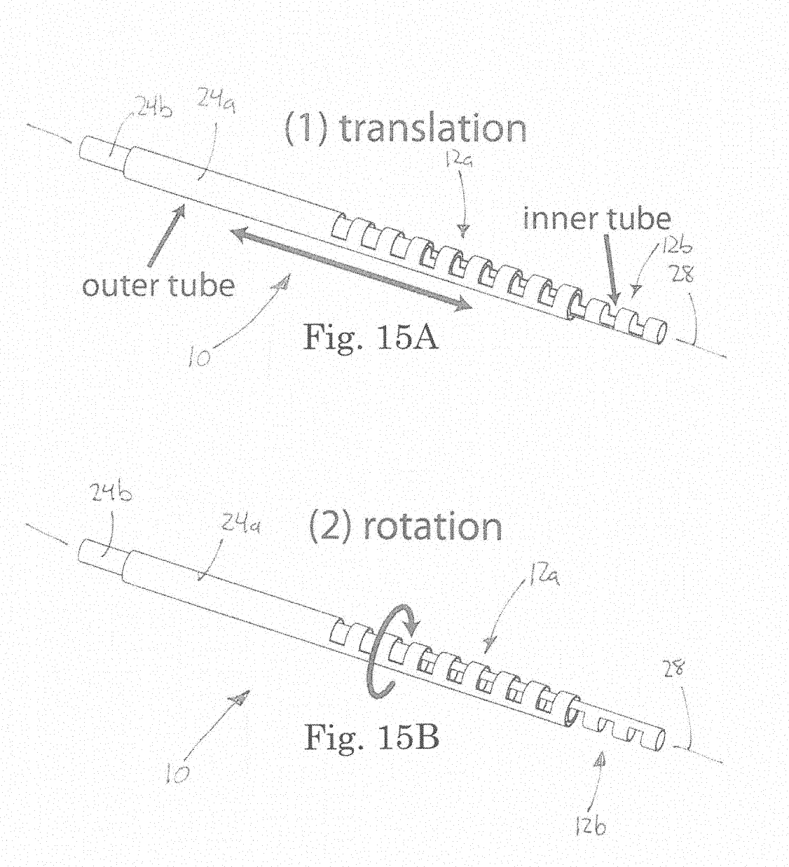

Referring to FIG. 15, the apparatus 10 could include multiple nested concentric tubes 24a, 24b, each of which includes its own corresponding bendable tip 12a, 12b. In this configuration, each bendable tip 12a, 12b can operate in accordance with any of the example embodiments described herein. For example, each bendable tip 12a, 12b can include one or more bend sections and corresponding tendon cables. As another example, the cutouts of either bendable tip 12a, 12b can be arranged in any radial configuration along the length of their corresponding tubes 24a, 24b.

Referring to FIG. 15, in operation, the nested concentric tubes 24a, 24b can be manipulated for (1) translation along the axis 28 and (2) rotation about the axis. Through this axial and translational manipulation, the tubes 24a, 24b can be advanced, rotated, and bent in order to follow a desired path and also to achieve a desired shape.

Device Modeling

To design the bendable tip 12 that exhibits an arc length varying curvature tailored to specific anatomical target structures and workspaces, kinematics and statics models are required. The kinematic model predicts the operation or motion of the bendable tip 12. The statics model predicts how forces acting on the bendable tip 12, i.e., the forces applied by the tendon cable 40, affect the bending of the bend joints 52 and sections 54.

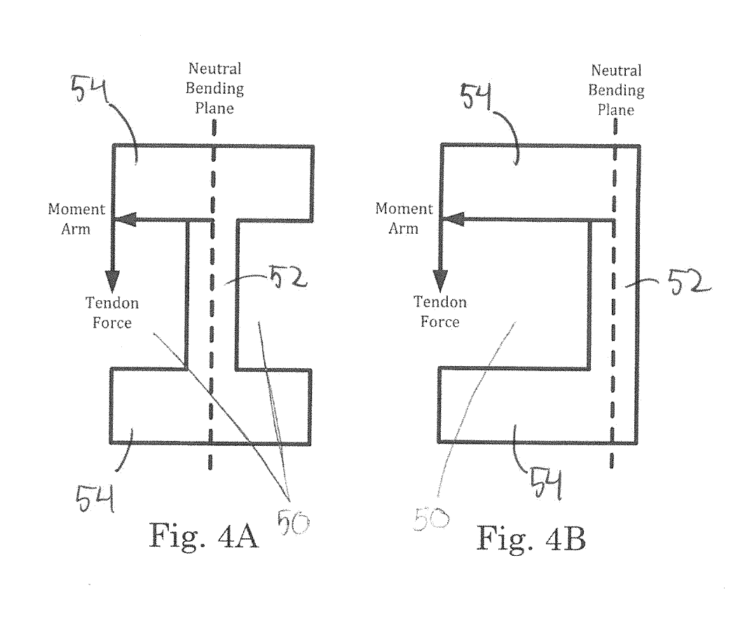

Referring to FIGS. 4A and 4B, the cutouts 50 can be either symmetric (FIG. 4A) or asymmetric (FIG. 4B). Of course, the asymmetric cutout 50 has a much longer moment arm for the tendon force. The designs illustrated in FIGS. 1-3 are asymmetric. One advantage of using asymmetric cutouts 50 is the longer moment arm between the tendon anchor point and the neutral bending plane, which enables significantly lower tendon cable 40 actuation forces for devices of comparable diameter. Another advantage is the ability to achieve a tighter radius of curvature, since the radius of curvature is measured about the center of the bendable tip 12, whereas the tip bends about an offset neutral bending plane. Other advantages of the asymmetric geometry include single wire actuation and simplified tendon routing, since the tendon will naturally conform to the inside wall of the tube when pulled, and one need not design mechanisms to hold it in place (e.g., the use of two nitinol tubes with the tendon sandwiched between).

One potential limitation of an asymmetric design is that it can bend in only one direction in the plane, rather than two. However, provided axial rotation of the entire device is possible (which it typically is for such devices), the impact of any potential drawback is minimized. Another potential limitation of an asymmetric bendable tip 12 is that while it can readily apply pulling forces, it can only apply pushing forces if the tissue being pushed is more compliant than the bendable tip 12 itself. It can, however, be possible to stiffen the bendable tip 12 to assist with pushing by inserting a wire through the central lumen.

In addition to being able to be manufactured and assembled at small diameters, the continuum cutout design also offers a large design space. In the kinematics and statics modeling, the cutouts 50 are restricted to rectangular cutouts because they are straightforward to machine. With this restriction, the design parameters available are the height, depth, and spacing between cutouts 50, as well as the number of cutouts. The models and design principles set forth below allow the designer to use these parameters to select the device's overall radius of curvature, total maximum bend angle, and required tendon force for actuation.

### Kinematic Modeling

We begin by modeling the kinematics of a single cutout of the asymmetric continuum bendable tip 12. We assume that the portion of the tube that undergoes bending deforms in a constant curvature arc. This is a good assumption for small cut heights h, because the tendon follows an approximately circular path in this case. Following the direction of R. J. Webster III and B. A. Jones, “Design and kinematic modeling of constant curvature continuum robots: a review,” The International Journal of Robotics Research, vol. 29, no. 13, pp. 1661-1683, 2010, we map tendon displacement (actuator space) to arc parameters (configuration space) then map arc parameters to task space.

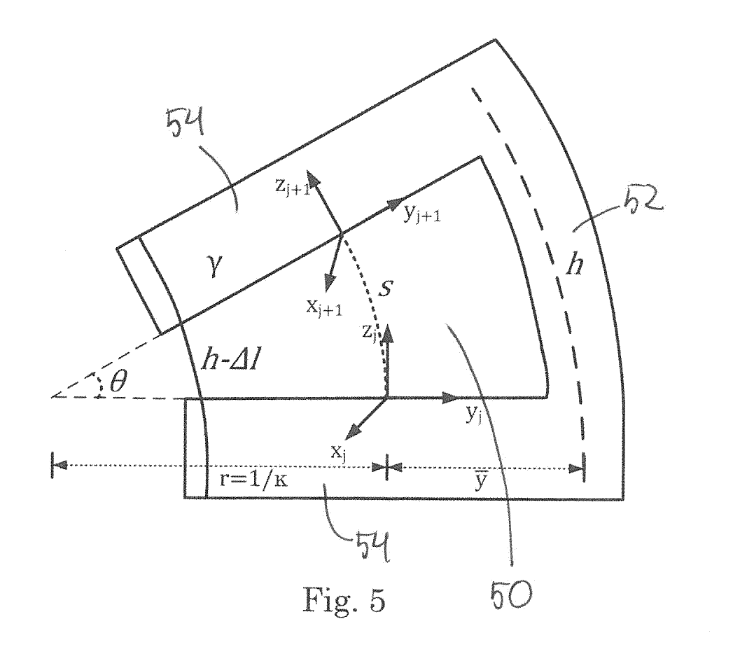

Arc parameters and relevant kinematic values for single cutout are shown in FIG. 5. The arc parameters we seek are curvature (κ) and arc length (s). The actuator space to configuration space mapping is largely dependent on the location y̅ of the neutral bending plane. The neutral bending plane experiences no strain in bending and intersects the centroids of the axial cross sections of the cut portions of the tube.

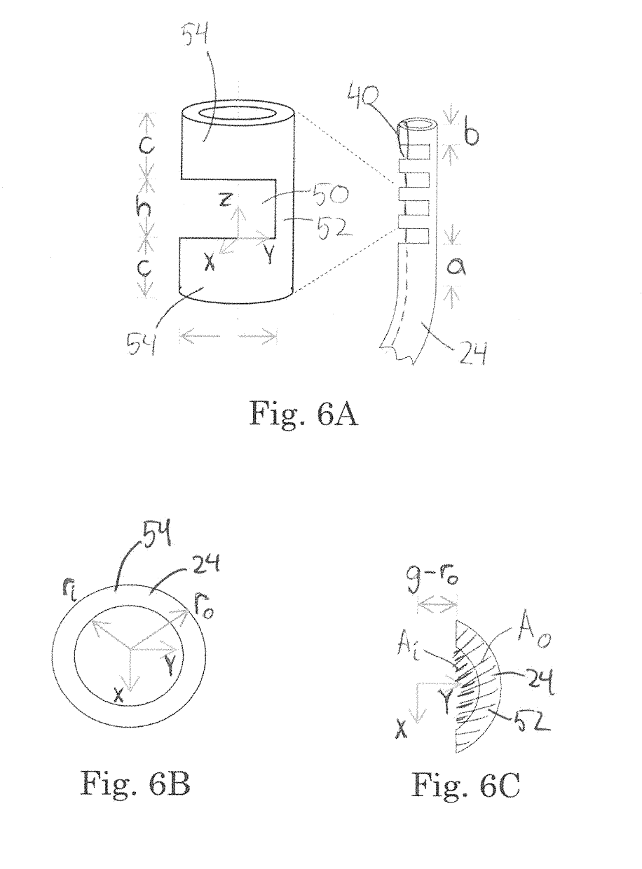

FIG. 6 illustrates the geometric parameters (a, b, c, g, h, rᵢ, rₒ) that the designer is free to choose. The tendon 40 is looped through the top cutout 50. The regions of the uncut portion of the tube used for the calculation of the neutral bending plane location are shown at Aᵢ and Aₒ.

The location of the neutral bending plane is dependent on the depth of cut g and the inner and outer radii of the tube (rᵢ and rₒ shown in FIG. 6) and is given by:

$$\overline{y} = \frac{{\overline{y}}_{o} {A}_{o} - {\overline{y}}_{i} {A}_{i}}{{A}_{o} - {A}_{i}} \qquad ( Eq . 1 )$$

where Ao and Ai are the areas defined in FIG. 6 and y̅ₒ and y̅ᵢ are their respective centroids. They are given by:

$${\overline{y}}_{o} = \frac{4 {r}_{o} {sin}^{3} ( \frac{1}{2} {φ}_{o} )}{3 ( {φ}_{o} - sin {φ}_{o} )} \\ {\overline{y}}_{i} = \frac{4 {r}_{i} {sin}^{3} ( \frac{1}{2} {φ}_{i} )}{3 ( {φ}_{i} - sin {φ}_{i} )} \\ {A}_{o} = \frac{{r}_{o}^{2} ( {φ}_{o} - sin ( {φ}_{o} ) )}{2} \\ {A}_{i} = \frac{{r}_{i}^{2} ( {φ}_{i} - sin ( {φ}_{i} ) )}{2} \\ {φ}_{o} = 2 arccos ( ( g - {r}_{o} ) / {r}_{o} ) \\ {φ}_{i} = 2 arccos ( ( g - {r}_{o} ) / {r}_{i} ) \qquad ( Eq . 2 )$$

which are valid for cuts that are at least as deep as the outer radius of the tube.

Now we can use y̅ to find the mapping from curvature to tendon displacement (Δl), noting FIG. 5 and using the chord function and arc geometry:

$$Δ l = h - 2 ( \frac{1}{k} - {r}_{i} ) sin ( \frac{κ h}{2 ( 1 + \overline{y} κ )} ) \qquad ( Eq . 3 )$$

Since we want the mapping of tendon displacement to curvature, we need to invert (3). Since it has no analytic inverse, numerical techniques can be used, or, for small angles, we can use a first-order approximation to yield:

$$κ \approx \frac{Δ l}{h ( {r}_{i} + \overline{y} ) - Δ l \overline{y}} \qquad ( Eq . 4 )$$

Once κ is known, s can be found using:

$$s = \frac{h}{1 + \overline{y} κ} \qquad ( Eq . 5 )$$

Once the arc parameters κ and s are known, the homogeneous transformation between frames j and j+1 (as defined in FIG. 5) can be found using:

$${T}_{j}^{j + 1} = [ \begin{matrix}1 & 0 \\ 0 & cos ( κ s ) \\ 0 & sin ( κ s ) \\ 0 & 0\end{matrix} \begin{matrix}0 & 0 \\ - sin ( κ s ) & ( cos ( κ s ) - 1 ) / κ \\ cos ( κ s ) & sin ( κ s ) / κ \\ 0 & 1\end{matrix} ] \qquad ( Eq . 6 )$$

Due to the rectangular cutout geometry of the bendable tip 12, the kinematic transformation from the base of the bendable tip 12 to the tip can be obtained. The kinematics of the entire bendable tip 12 are given by repeatedly applying the transformation (6) in conjunction with translations to account for the portions of the bendable tip 12 that do not bend:

$${T}_{o}^{t} = {T}_{z , a} ( {\prod }_{j = 1}^{n} {T}_{j}^{j + 1} {T}_{z , c} ) {T}_{z , b - c} \qquad ( Eq . 7 )$$

where n is the number of cutouts and T_{z-a}, T_{z,b-c}, and T_{z,c} are translations along the z-axis by a, b-c, and c, respectively, as defined in FIG. 6. In addition, the angle of rotation of each section can be found explicitly as:

$${θ}_{j} ( κ ) = s κ = ( \frac{h}{1 + \overline{y} κ} ) κ \qquad ( Eq . 8 )$$

And thus the maximum angle of rotation for a single cutout is given by:

$${θ}_{j , max} = {θ}_{j} ( 1 / {r}_{o} ) = \frac{h}{{r}_{o} + \overline{y}} \qquad ( Eq . 9 )$$

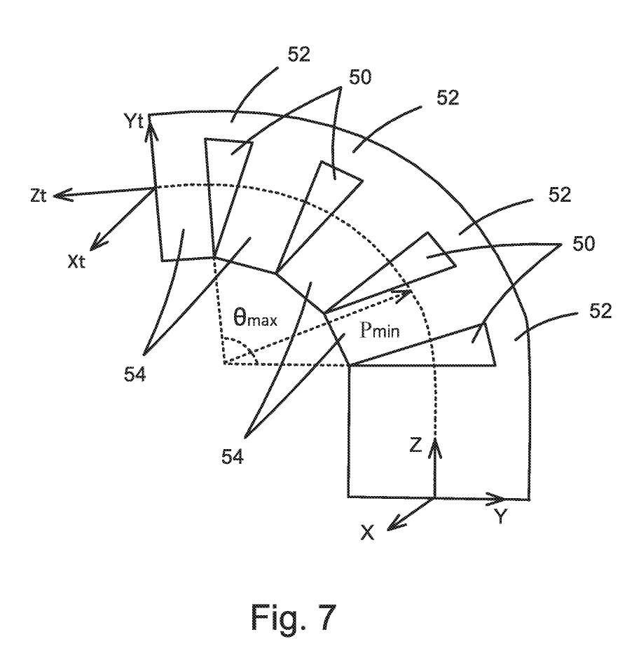

Two important bendable tip 12 characteristics, maximum bending angle and minimum radius of curvature, as shown in FIG. 7, can be calculated from geometry as:

$$\begin{matrix}{θ}_{max} = {\sum }_{j = 0}^{n} {θ}_{j , max} = n \frac{h}{{r}_{o} + \overline{y}} & ( Eq . 10 ) \\ {ρ}_{min} \approx {r}_{o} + \frac{( n - 1 ) c}{{θ}_{max}} & ( Eq . 11 )\end{matrix}$$

where the approximately circular arc that defines ρₘᵢₙ has length:

$$S = n ( \frac{{r}_{o} h}{{r}_{o} + \overline{y}} + c ) - c \qquad ( Eq . 12 )$$

### Statics Modeling

Modeling the static behavior of the bendable tip 12 is more challenging than modeling the kinematic behavior, yet with the assumption of constant curvature bending, it is tractable. Based on the constant curvature assumption, strain along the length of the bendable tip 12 varies in a cross section of the portion of the tube in bending according to:

$$ϵ ( y , κ ) = \frac{κ ( y - \overline{y} )}{1 + \overline{y} κ} \qquad ( Eq . 13 )$$

and thus is linearly distributed about the neutral bending plane. This assumed relationship between the geometry and the material deformation allows for a simple computation of the strain energy, after which we use Castigliano's first theorem to determine the reaction force at the tendon. In general, the behavior of nitinol under applied stresses is complex and highly nonlinear, and depends on thermomechanical history. In this work we assume a simplified material model that represents the stress-strain behavior of nitinol as a piecewise linear stress-strain curve, so that the stress may be written as a function of strain as:

$$σ ( ϵ ) = \{ \begin{matrix}{σ}_{lp} \\ {E}_{ϵ} \\ {σ}_{up}\end{matrix} \begin{matrix}ϵ < {σ}_{lp} / E \\ {σ}_{lp} / E \leq ϵ \leq {σ}_{up} / E \\ ϵ > {σ}_{up} / E\end{matrix} \qquad ( Eq . 14 )$$

where σₗₚ is the lower plateau stress (corresponding to compression), σᵤₚ is the upper plateau stress (corresponding to tension), and E is Young's modulus. Since we are modeling the material deformation as a one-dimensional stretching and compression of axial fibers, the strain energy density is the area under the stress-strain curve, given by the integral:

W(∈)=∫₀^{∈}σ(e)de (Eq. 15)

The total strain energy stored in the bendable tip 12 as a function of the curvature κ of a single cutout is given by:

U(κ)=n∫_{V}_{_{c}}W(∈(y,κ))dV (Eq. 16)

where V_{c} is the volume defined by the “Top View Cut” cross section of FIG. 6 and cutout height h. We use Castigliano's first theorem to find the relationship between rotation θ of the bendable tip 12 and force F applied by the tendon to the bendable tip 12 tip:

$$\frac{\partial U ( κ )}{\partial θ} = M = FL \qquad ( Eq . 17 )$$

where L is the moment arm length and θ=nsK. When the tendon is looped around the top flexure as shown in FIG. 6, the moment arm has length

$$L = ( \frac{{r}_{o} + {r}_{i}}{2} ) + \overline{y} .$$

Due to friction, the force the tendon applies to the tip of the bendable tip 12 will be a fraction of the actuator force applied to the tendon. Friction between the tendon and the tube wall becomes increasingly significant as cut height and angle of bending increase. To model this effect, we first find the angle γ (shown in FIG. 5) that the tendon is required to navigate at a single corner of a cutout section at a given angle of deflection. We assume that the friction that occurs at these corners dominates friction elsewhere along the tendon path. Writing the static balance equations for a single corner, with _s as the static friction coefficient, we find that:

$$F = {ηF}_{tendon} = \frac{sin γ / 2 - {μ}_{s} cos γ / 2}{sin γ / 2 + {μ}_{s} cos γ / 2} {F}_{tendon} \qquad ( Eq . 18 )$$

where η<1 accounts for the force lost due to friction at a corner. We can substitute (18) into (17) to yield:

$${F}_{tendon} = \frac{1}{{η}^{2 n} L} \frac{\partial U ( κ )}{\partial θ} \qquad ( Eq . 19 )$$

where 2n is included to account for the two corners of each cutout. This expression can be evaluated numerically using a finite difference method to relate F_{tendon} and θ. This statics model is experimentally validated in the following paragraphs.

### Prototype and Experimental Validation

A prototype of the bendable tip 12 is shown in FIGS. 8, 9, and 10. The prototype bendable tip 12 carries a curette as the surgical instrument. The curette is connected to a nitinol wire that runs through the tube. The wire can be rotated to effectuate rotation of the curette.

The prototype bendable tip 12 was built using a MicroProto Systems MicroMill 2000 CNC mill (a small tabletop CNC mill) with aluminum titanium nitride coated, two flute, carbide, long flute, 0.02″ diameter square end mills. The tube was fixtured by gluing it in a channel drilled in an aluminum block. The nitinol tube had an outer diameter 1.16 mm and inner diameter of 0.86 mm. A cut depth of g=0.97 mm was chosen, which corresponds to a required tendon force for full bending of F_{tendon}=5N and a maximum outer-fiber strain of 10.4% (Note that this is slightly higher than the 8-10% recoverable strain typically quoted for nitinol, but that it has been found to work well in practice, since only a small amount of the material at the very outside edge of the bendable tip 12 undergoes this strain, and then only at maximum articulation). The cut height was h=0.51 mm. The spacing between cuts was c=0:51 mm. The number of cuts was n=5 cuts in order to achieve greater than 90 degrees of bending. A summary of the design parameters and resulting design characteristics is shown in Table I:

| Parameter | Value | Characteristic | Value |
|---|---|---|---|
| Dₒ | 1.16 mm | θₘₐₓ | 138.6° |
| Dᵢ | 0.86 mm | ρₘᵢₙ | 1.42 mm |
| g | 0.97 mm | εₘₐₓ | 10.4% |
| h | 0.51 mm | F_{tendon} | 5 N |
| c | 0.51 mm | | |
| n | 5 | | |

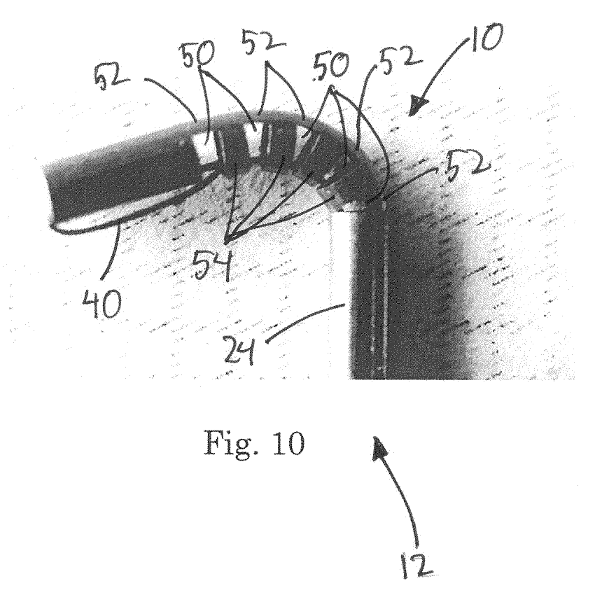

FIG. 7 illustrates the motion of the bendable tip 12 motion from 0 to 90 degrees bending angle. Note that the ring curette is being rotated during the bending motion of the bendable tip 12. The prototype was experimentally validated without the curette instrument attached (see FIG. 10). The actuation tendon held open the last cutout of the bendable tip 12 during the experimental trials, and the kinematics and statics models were calculated with n=4 cutouts.

An experiment was conducted to validate the kinematic relationship of Equation 7 and the static relationship of Equation 19 concurrently. The experimental setup included a linear slide (Velmex A2512Q2-S2.5) with 0.01 mm resolution to displace the tendon and a force sensor (ATI Nano 17) with 3.125 mN resolution to record tendon force. The tendon was rigidly fixed to an acrylic plate that was then mounted onto the force sensor. The tendon and sensor assembly were then rigidly fixed to the linear slide carrier.

The nitinol tube with cutout bendable tip 12 was mounted into a test fixture that was rigidly mounted to an optical table, such that the tube remained stationary while the bendable tip 12 was deflected with the linear slide. A 1 mm resolution grid was placed below the bendable tip 12, and a camera mounted directly above the bendable tip 12 was used to capture images of the bendable tip 12 as it deflected. The bendable tip 12 was deflected in tendon displacements of 0.2 mm, and a picture of the bendable tip 12 deflection and the tendon force were recorded at each increment.

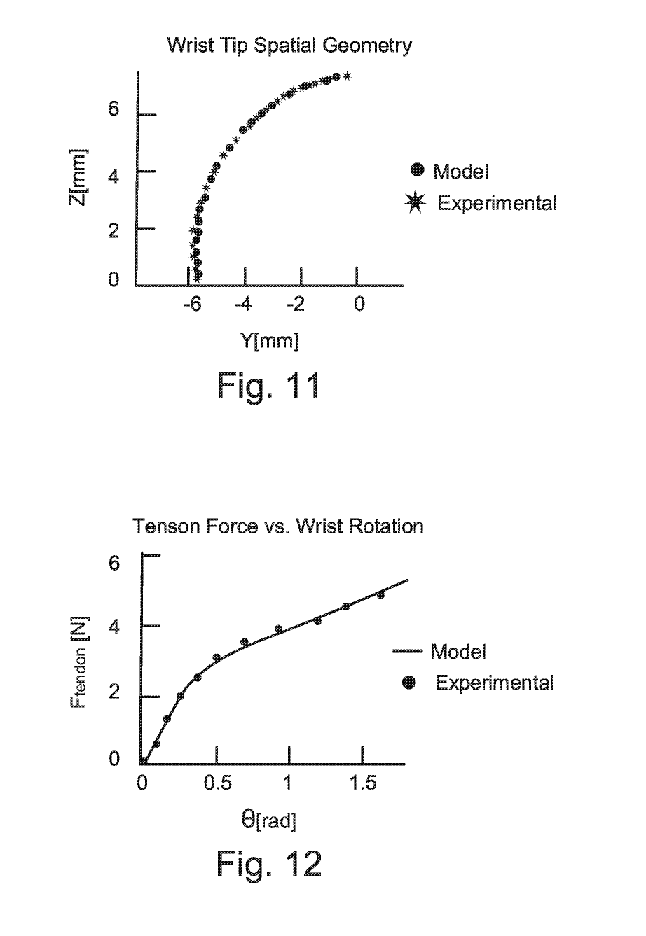

Using image processing, the tip position was determined for each incremental deflection of the tendon. At full articulation, it was observed that the distal cutout was held open by the tendon that was routed through it (see FIG. 10). For this reason, the plots in FIGS. 11 and 12 were made based on n=4 cutouts. Alternative tendon attachment methods can address this issue. Results of the experiment are shown in FIGS. 11 and 12.

Referring to FIG. 11, the bendable tip 12 starts at top of the figure and rotates counterclockwise from 0 to 110 degrees. These results show that the constant curvature assumption is a reasonable approximation for this geometry, since the bendable tip 12 tip follows the path predicted by the model.

An experimental validation of the statics model is shown in FIG. 12. The model captures the superelastic behavior of the material, with the change in the slope of the graph indicating the transition of some of the volume of material into the stress plateau region. For the material properties, note that nitinol has an asymmetric stress strain relationship in tension and compression. We assume plateau stresses of σᵢₚ=−750 MPa and σᵤₚ=500 MPa and a Young's modulus of E=60 GPa, which fall within ranges reported by the manufacturer and in the literature. The model is shown with a coefficient of friction of 0.36, which was chosen through nonlinear least squares optimization. Note that the superelastic, nonlinear behavior of the material is clearly captured by the model.

### DISCUSSION

The prototype represents one set of viable design choices. With the rectangular cut profile described in previous sections, the designer must choose the depth of cut g (see FIG. 6), height of cut h, number of cuts n, and axial spacing between cuts c. Moreover, the designer also has some freedom to select the tube radii, though this is likely to be from among a finite set of options due to material availability. The tube radii and the depth of cut are the most important parameters in determining bendable tip 12 behavior, because they determine the location of the neutral bending plane, which strongly affects the kinematics, strain in the bending material, and the required actuation force. A cut depth g>rₒ is desirable to achieve substantial bending compliance. The allowable depth of cut is bounded by the maximum allowable strain, where the maximum strain at full bending is given by:

$${ϵ}_{max} = ϵ ( {r}_{o} , 1 / {r}_{o} ) = \frac{{r}_{o} - \overline{y}}{{r}_{o} + \overline{y}} \qquad ( Eq . 20 )$$

Cut height is not as significant as cut depth in determining bendable tip 12 behavior, but it is a factor in the bending radius (Eq. 10 and 11). Moreover, if cut height becomes too large, the constant curvature assumption will no longer hold, risk of buckling-like failure will increase, and frictional losses will increase (Eq. 18 and 19).

The portions of uncut tube between the cutouts serve as hard stops which limit strain, allow large forces to be applied in the bendable tip's fully deflected state, and route the tendon in a curve that approximates a circular arc. The height of the uncut portions, parameter c in FIG. 6, should be as small as possible to minimize radius of curvature. However, as it decreases, risk of damaging the bendable tip 12 during actuation and environmental interaction increases.

Additionally, if uniform curvature in multiple cutout sections is desired, it is essential to use a highly repeatable cutting process, as slightly deeper cutouts deflect much further for a given force than shallower cutouts do. That being said, it may be advantageous in future work to take advantage of non-uniform cut depths (and/or heights) to compensate for factors like non-constant tendon tension (due to frictional losses) along the bendable tip 12, or application-specific design objectives.

The experimental results show that the constant curvature assumption is a reasonable, though not perfect, approximation for our bendable tip 12. We believe that tendon elongation was the primary source of error in the kinematics, which resulted in the model and experimental tip points not aligning perfectly in FIG. 11. The coefficient of friction is likely the least well known of all the parameters, since the amount of friction depends on factors such as surface roughness and geometry. Another potential source of error is the implicit assumption that cross sections do not deform during bending, which is a common assumption in beam bending analysis.

In the future, we plan to study the significance of hysteresis in our statics model and develop a three-dimensional stiffness model in order to characterize the forces that the bendable tip 12 can exert. We also plan to conduct finite element modeling to characterize torsional properties and fatigue life and to explore strain profiles of non-rectangular cutouts. Another area of future work is to explore non-square cutout geometries to optimize bendable tip 12 performance for specific tasks.

### Steerable Needle Bendable Tip

Another embodiment employing the same principles described above is illustrated in FIGS. 16-18. Referring to FIGS. 16A and 16B, in this embodiment, the bendable tip surgical device 12 is a bendable tip steerable needle 110. The steerable needle 110 is a needle constructed of a flexible tube 112 with a one or more cutouts 114 that create a compliant bending region 116 of the tube. The number of cutouts 114 can vary depending on the desired bending performance characteristics for the needle 110. In the embodiment illustrated in FIGS. 16-18, the needle 110 includes a single cutout 114. The needle 110 could, however, include multiple cutouts 114 and perform in accordance with the descriptions of embodiments set forth above in which the bendable tip includes multiple cutouts. As an example, the flexible tube 112 can be constructed of a nickel-titanium, i.e., “nitinol,” alloy.

The cutout 114 defines the boundary between an elongated body portion 120 and a tip 122 of the steerable needle 110. The body portion 120 can have any desired length, which can, for example, vary depending on the procedure in which the steerable needle 110 is implemented. The tip 122 is formed by a beveled cut of the tube 112 that is filled or closed off, for example, by welding, soldering, or brazing, to form an angled or beveled lead surface 124. Alternative fillers, such as a polymer, can be used to fill the tip 122 and form the lead surface 124.

The cutout 114 extends into the tube 112 in a direction normal to the tube axis 130, entering the tube from opposite the lead surface 124. In the embodiment illustrated in FIGS. 16-18, the cutout 114 has a generally rectangular cross-section. It is the dimensions of the cutout 114 and the diameter of the tube 112 that determine the range, indicated generally by angle θ, that the tip 122 can bend relative to the body portion 120. This range of bending can be controlled or configured through the selection of the dimensions of the cutout 114, e.g., the width of the cutout as measured along the axis 130. The range of bending can also be controlled or configured through the selection of the shape of the cutout 114, e.g., a V-shaped cutout as opposed to a rectangular cutout.

The elongated tubular configuration of the bendable tip needle 110 advantageously includes a long inner lumen that defines a channel 126 within the body portion 120 of the tube 112 that extends to the opening, i.e., the cutout 114, adjacent the needle tip 122. This channel 126 can serve as a large working channel from the base of the needle to the tip, for example, to perform biopsy or drug delivery therapies. Further facilitating this is the fact that the bend is facilitated by the cutout 114 in the tube 112, which eliminates the need for any mechanical joint components that would consume space in the channel 126.

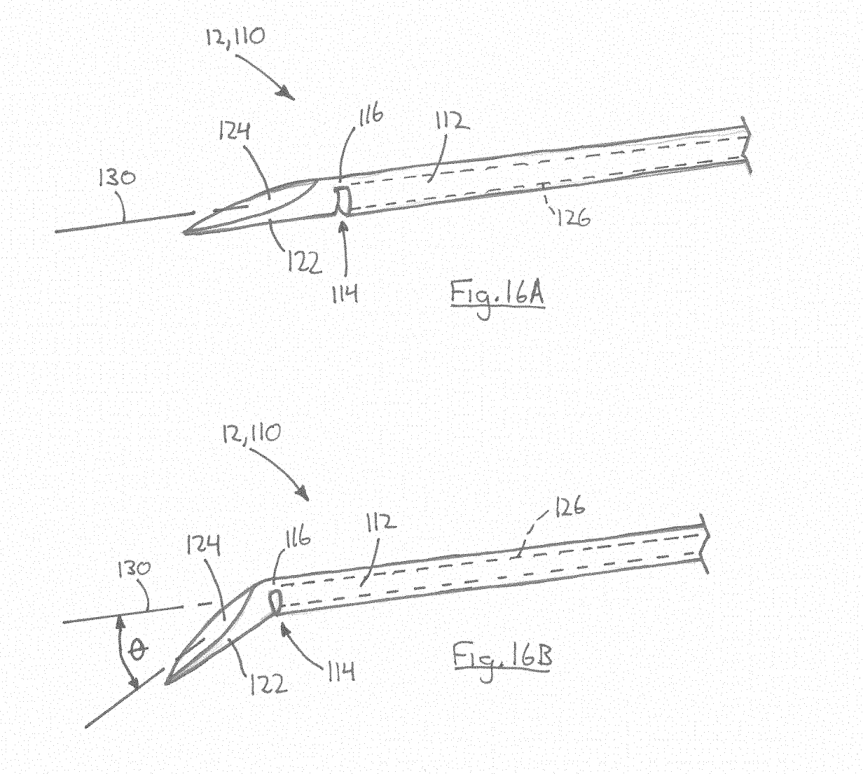

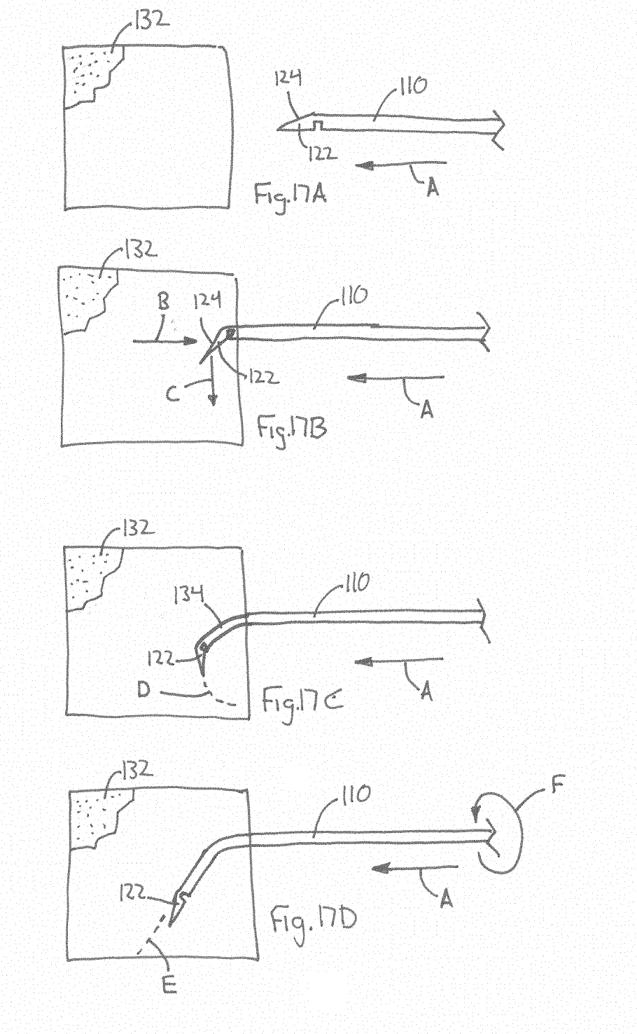

Referring to FIGS. 17A-17D, in operation, the steerable needle 110 is advanced toward a body of tissue 132, such as human body tissue, in a direction indicated generally by arrow A. As the needle 110 enters the tissue 132, the tissue offers resistance to needle advancement, as indicated generally by the arrow B in FIG. 17B. A component of these resistance forces act normal to the lead surface 124 of the needle tip 122, indicated generally by arrow C. These component forces cause the tip 122 to bend relative to the body portion 120 in a manner described above in regard to FIGS. 16A and 16B.

Referring to FIG. 17C, the bent tip 122 causes the needle 110 to follow a curved path, as indicated generally by dashed line D in FIG. 17C. As a result, a portion 134 of the body portion immediately trailing the tip 122 follows this curved path and assumes a curved configuration.

Referring to FIG. 17D, when the tip 122, following the curved path D, reaches a desired trajectory, the body portion 120 can be rotated, as indicated generally by the arrow F in FIG. 17D, which causes the tip to resume its non-bent configuration, extending along a path E that is coaxial with the body portion. Maintaining this rotation while the needle 110 is advanced (arrow A) can cause the needle to follow a straight path. When rotation is stopped, the tip 122 will again bend and follow a curved path as the needle is advanced further. Thus, by selecting the rotational orientation of the lead surface 124, the curved path, i.e., the curved direction of needle advancement, can be selected.

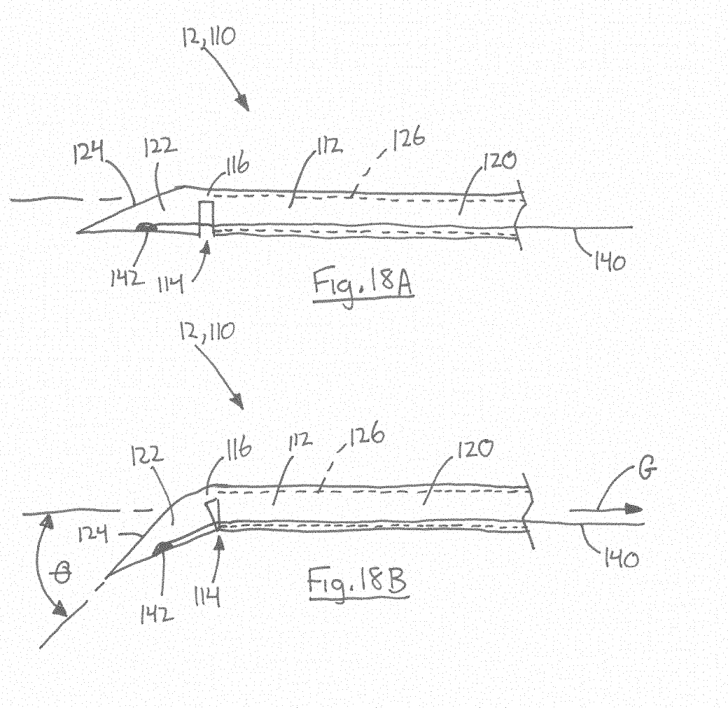

Referring to FIGS. 18A and 18B, the large working channel 126 of the steerable needle 110 can be used to house an actuating member 140, such as a cable or wire (e.g., nitinol wire), that acts as a tendon for actuating the bendable tip 122. The tendon cable 140 is connected to the interior of the tip 122 at a connection point 142 formed, for example, via weld, solder, brazing, or adhesive bond. Tension on the tendon cable 140, as indicated generally by the arrow G in FIG. 18B, causes the tip 122 to bend at the bending portion 116.

In the configuration of FIGS. 18A and 18B, the tube material can be selected and the bendable tip 122 can be configured such that the tip will not bend in response to tissue forces acting on the lead surface 124 as described above. Instead, bending of the tip 122 can be controlled primarily or exclusively through tension applied via the tendon cable 140. In this manner, the degree of tip deflection, and thus the amount of curvature with which the needle 110 responds, can be selected through the displacement of the tendon cable 140. Thus, not only does the tendon cable 140 allow for precise control of when the tip 122 bends, but also the degree to which it bends. As a result, the configuration of the bendable tip steerable needle 110 in FIGS. 18A and 18B can allow for a higher degree of precision in steering the needle.

The bendable tip steerable needle 110 is suited for any needle-based procedure that requires accurate targeting and also provides the ability to reposition/retarget without full removal of the needle. This feature can be particularly useful, for example, for correcting needle misalignment or unforeseen deflection of the needle during insertion.

The design of the bendable tip steerable needle 110 is straightforward and simple to build from a manufacturing perspective, while advantageously leaving the center working channel open all the way to the tip of the needle. Tip deflection can be achieved in a simple, accurate, and repeatable manner through tension on the tendon cable 140. Though simple in design, the steerable needle 110 can exhibit a high degree of steerability with minimized tissue damage and a high degree of curvature.

We claim:

1. An apparatus for performing surgery on a patient, comprising:
a tubular structure comprising a tubular side wall that extends along an axis and defines an inner lumen, wherein a distal end of the tubular structure is configured to perform a surgical function;
wherein the tubular structure comprises a plurality bend joints that are deflectable so that the portion of the tubular structure along which the bend joints lie assumes a curved configuration; and
wherein the apparatus further comprises a plurality of actuating members, each actuating member being connected to the tubular structure at a location radially opposite a corresponding one of the bend joints, and each actuating member being individually actuatable to apply tension that causes the bend joints proximal to its connection to the tubular structure to deflect and assume the curved configuration, which creates a bend in the tubular structure.

2. The apparatus recited in claim 1, wherein each bend joint is at least partially defined by a cutout in the tubular side wall, wherein each cutout comprises an axial portion of the sidewall that is removed and provides communication with the inner lumen, the bend joints comprising the remaining portions of the side wall left along the length of the cutout, and wherein the cutouts at least partially define at least one bend section comprising complete tubular portions of the sidewall on opposite sides of the cutouts.

3. The apparatus recited in claim 2, wherein the actuating members are connected to the bend sections.

4. The apparatus recited in claim 1, further comprising an end effector connected to the distal end of the tubular structure, the end effector being configured to perform the surgical function.

5. The apparatus recited in claim 1, wherein the plurality of actuating members are configured to be tensioned simultaneously to create a compound bend in the tubular structure.

6. The apparatus recited in claim 5, wherein the plurality of actuating members are configured to be tensioned sequentially to create the compound bend.

7. The apparatus recited in claim 1, wherein the cutouts are configured so that the bend sections are arranged in a pattern along the length of the tubular structure, the actuating members when actuated creating bends in the tubular structure that follow the pattern.

8. The apparatus recited in claim 1, wherein the cutouts are configured in multiple groups each comprising multiple cutouts, the bend sections of each group being arranged in a pattern along the length of the tubular structure such that the actuating members when actuated create bends in the tubular structure that follow the patterns of the groups.

9. The bendable joint recited in claim 8, wherein the cutouts of at least one group are rotated relative to each other along the axis of the tubular structure.

10. The apparatus recited in claim 1, wherein the cutouts are configured so that the bend sections are aligned linearly with each other parallel to the axis of the tubular structure, the actuating members being actuatable independently to create multiple bends in the same bending plane.

11. The apparatus recited in claim 1, wherein the cutouts are configured in groups of multiple cutouts, the bend sections of each group being aligned with each other parallel to the axis of the tubular structure, and the bend sections of each group being positioned at different radial positions with respect to the axis, the actuating members being actuatable independently to create multiple bends in different bending planes.

12. An assembly comprising a plurality of apparatuses as recited in claim 1, wherein the tubular structures of each apparatus are arranged in a nested, concentric, and coaxial manner.

13. The apparatus recited in claim 12, wherein bends created in one of the tubular structures due to tensioning one or more of the actuating members creates a bend in the coextending portion of the other concentric tubular structures.

14. The bendable joint recited in claim 1, wherein the cutouts have geometries selected such that the physical properties of the bend joints differ from each other, which causes the curvature of the bend joint to vary along its length.

15. The bendable joint recited in claim 1, wherein the cutouts have geometries selected such that the physical properties of the bend joints differ from each other, which causes the bend joints to deflect in a predetermined order in response to tension applied to the actuating member.

16. The bendable joint recited in claim 1, wherein the tubular structure comprises an inner tube of a concentric tube robot.

17. The bendable joint recited in claim 1, wherein the cutouts have rectangular geometries.

18. The bendable joint recited in claim 1, wherein the geometries of the bend sections defined by the cutouts are configured to define the amount of deflection that each bend joint can undergo.

19. The bendable joint recited in claim 1, wherein the geometries of the bend sections defined by the cutouts are configured to collectively define the range of bending motion that can be achieved by the bendable joint.

20. The bendable joint recited in claim 1, wherein the tubular structure comprises a nitinol tube.

21. The bendable joint recited in claim 4, wherein the end effector comprises a needle tip comprising a sharpened point.

22. The bendable joint recited in claim 21, further comprising a tendon cable that extends within the inner lumen and has a connection with the needle tip, wherein tension on the tendon cable is applied to the needle tip to cause bending at the needle tip.

23. The bendable joint recited in claim 4, further comprising a tendon cable that extends through the tubular structure and is connected to the end effector, the tendon cable being actuatable to cause actuation of the end effector.
